(19) United States
(12) Patent Application Publication
(10) Pub. No.: US 2015/0367112 A1
(43) Pub. Date: Dec. 24, 2015

(54) Medical Balloon with Reduced Straightening Force

(71) Applicant: Daniel Gelbart, Vancouver (CA)

(72) Inventor: Daniel Gelbart, Vancouver (CA)

(21) Appl. No.: 14/457,311

(22) Filed: Aug. 12, 2014

Related U.S. Application Data
(60) Provisional application No. 62/014,696, filed on Jun. 20, 2014.

(51) Int. Cl. A61M 29/02; B29C 53/56

(52) U.S. Cl. CPC A61M 29/02; B29C 53/566; B29K 2077/10

(57) ABSTRACT

A high pressure balloon uses a shallow helical groove embossed in the balloon wall to decrease the straightening force of the balloon. A filament made from a high tensile strength material with low compliance is wrapped in the embossed groove to prevent internal pressure within the balloon from flattening the groove.

### TECHNICAL FIELD

The invention relates to balloons, or balloon catheters, for use in medical procedures such as stent deployment, angioplasty, heart valve placement, aortal graft placement and other procedures requiring high-pressure balloons.

### BACKGROUND

Medical balloons, particularly, those used in the vascular system, should satisfy several opposing requirements. Since balloons are typically introduced through small diameter sheaths, it is desirable to make balloons from very thin material to facilitate folding the balloons to small diameters. However, such balloons are often operated at high pressures, typically from 10 to 20 atmospheres, which requires the balloons to be made from high tensile strength materials such as Nylon12, polyurethane, PET or PEBAX. Balloon materials should have a limited ability to stretch, as the shape of the balloon should follow the designed shape and not be affected by the resistance it encounters while expanding.

While meeting all these requirements, a balloon should be flexible both in a deflated (folded) state and in a fully-inflated state. Flexibility when folded is referred to as “crossing profile” or “trackability”. This property allows a balloon to follow a tortuous vascular path (e.g. following a guidewire through vascular passages). Trackability can be achieved by using very thin materials for the balloon wall with proper folding. For this reason, medical balloons are typically made of material having thickness in the range of 10 to 30 microns. A balloon should also have a low straightening force when fully inflated, as the balloon may be placed in a bent part of an artery or vein when inflated. Forcing the artery or vein to straighten, even momentarily, can cause trauma and can lead to stenosis.

Unfortunately, the same qualities that have been exploited to allow balloons to have good trackability and well defined shape tend to give the balloons a strong straightening force when inflated. This is illustrated in FIG. 1 which is a schematic view of a prior art balloon 1. Balloon 1 has a very thin wall 4. Balloon 1 has an inflation tube 2 and a guidewire passage 3. Balloon 1 is normally straight. If one attempts to bend balloon 1 to form a curve the portion 4′ of wall 4 on the inside of the curve tends to become pushed in or wrinkled because the wall material has a very limited elastic range, typical of the high tensile strength materials used. For this to happen the volume of the balloon would have to decrease. Since such balloons are typically inflated by a non-compressible fluid (e.g. saline) using a positive displacement piston pump, any change that would reduce the volume of the balloon is resisted by increased fluid pressure within the balloon. As a result, the balloon resists bending with a straightening force which tries to keep the balloon straight with a circular cross section. For a balloon to have good bending flexibility (low straightening force) when pressurized, its volume should not change while the balloon is being bent.

Prior art attempts to create a more flexible balloon can be grouped into five categories, as illustrated in FIGS. 2A to 2E. The simplest idea is to make the balloon from a softer material that is more elastic (i.e. a material that has a smaller modulus of elasticity). This is illustrated symbolically in FIG. 2A. The problem is that the balloon will not have a controlled shape. It will expand according to the resistance it encounters and also will elongate significantly with inflation, as the longitudinal stress is a significant portion of the hoop (circumferential) stress.

Balloons are classified as “compliant” (elastic) or “non-compliant” (inelastic), which are relative terms. Any material has an elastic range. When this range is exceeded material can either break or undergo a plastic deformation. Non-compliant balloons typically have an elastic range of under 20%, and even compliant balloons used in vascular procedures have a limited elastic range, typically 20%-40%.

FIG. 2B shows an example of a compliant balloon wrapped with a less compliant filament 5. This general structure is disclosed in several US patents and patent applications including 2014/0172066, 2010/0318029, 2007/0112370, 2004/006359, U.S. Pat. Nos. 8,672,990, 8,349,237, 8,221,351, 8,105,275, 8,002,744, 7,914,487, 6,824,553, 6,773,447, 5,772,681, 4,498,473, 8,486,014, 2010/0286760, 2011/0029064, U.S. Pat. Nos. 7,803,180, 6,878,162, 2002/0103529, U.S. Pat. No. 5,449,373, 2014/0135891, U.S. Pat. Nos. 5,569,220, 5,171,297, 6,245,040, 6,626,861, 7,001,420, 6,641,603, and others. Such a balloon is usable only at relatively low inflation pressures as the more elastic materials have a significantly lower tensile strength. The wound filament does not increase the longitudinal strength. If the filament is wound in a two-dimensional mesh pattern, to increase longitudinal strength, the material becomes non-compliant and stronger, but loses its flexibility.

FIG. 2C shows another example balloon in which deep grooves 6 are formed in the wall of a non-compliant balloon to increase flexibility. Balloons having this general configuration are described in U.S. Pat. Nos. 8,257,418 and 5,545,132. While grooves 6 may improve the crossing profile and blood flow during deployment, such embossing disappears when the balloon is fully pressurized as the thin wall (10 to 30 μm typically) cannot keep the embossed shape against the high pressure, typically 10 to 20 atm, which tries to maximize balloon volume. This fact is clearly stated in both these patents (page 2 lines 45-51 in U.S. Pat. No. 8,257,418 and page 5 lines 20-36 in U.S. Pat. No. 5,545,132). Shallow embossing is also used in a commercial product, the Rival™ balloon made by Bard Vascular (Tempe, Az.), but this embossing also disappears when the balloon is fully pressurized. The embossing is provided to improve trackability when the balloon is deflated. Deep grooves were achieved in U.S. Pat. No. 4,762,130, wherein the grooves themselves were achieved by wrapping an inflatable helical balloon around a catheter

FIG. 2D shows a non-compliant balloon divided into longitudinal segments. U.S. Pat. Nos. 6,048,350, 6,776,771, 7,658,744, 7,740,609, and 6,022,359 show examples of this construction. Expanding a stent or artery with such a balloon leads to an imprint in the shape of the segments which is highly undesirable. The transition between segments is wide, as a tapered section is needed at the ends of each segment. Without a tapered section the stress is too high.

FIG. 2E shows another prior art balloon. The balloon is divided longitudinally into several smaller balloons. U.S. Pat. No. 8,758,386 provides an example of this construction. This balloon can have good performance but it is undesirably expensive to manufacture.

There remains a need for medical balloons that provide low straightening force and yet are cost effective to manufacture.

### SUMMARY OF THE INVENTION

This invention has a number of aspects including balloons in a variety of embodiments, methods for making such balloons and methods for using such balloons.

A balloon according to an example embodiment has a shallow helical groove formed in the balloon wall. Such a balloon may be designed for use at high inflation pressures (e.g. pressures of at least 5 atm). In some embodiments the balloon is designed for inflation pressures in the range of 10 to 20 atm. The groove increases the bending flexibility of the balloon especially when the balloon is inflated. A filament made from a high tensile strength material with low compliance wraps around the balloon in the embossed groove and is affixed within the groove. The filament prevents the internal pressure from flattening the groove when the balloon is inflated. In some embodiments ends of the balloon are connected together by a flexible member that extends within the balloon.

The walls of medical balloons according to some embodiments are made from non-compliant (or limited compliance) materials. Limited compliance materials that are commonly used today for making balloons (e.g. Nylon 12, PET, PEBAX, polyurethane etc.) are examples of such materials.

The design of balloons according to example embodiments permits the balloons to retain high bending flexibility when inflated to high pressures and yet to maintain a crossing profile at least as good as standard balloons when deflated.

Methods according to some embodiments provide ways to manufacture balloons characterized by a low straightening force using equipment similar or identical to the equipment currently used to make medical balloons. In some embodiments the balloons can have pressure ratings above that of a standard balloon made from the same material and wall thickness.

Further aspects and example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 is a side view of a prior art bent balloon, showing volume change.

FIGS. 2A through 2E illustrate various prior art balloons.

FIG. 3 is a side view of a balloon according to an example embodiment of the invention.

FIG. 4A is a cross section of a wall of a balloon without pressure and FIG. 4B shows the effect of pressure on the balloon wall of FIG. 4A. FIG. 4C shows a possible embodiment which comprises an external sheath.

FIG. 5 is a cross section of a molding tool which may be used to manufacture balloons of the type shown in FIG. 3.

FIG. 6 is a side view of an example balloon incorporating a cord to limit longitudinal expansion.

FIGS. 7 A and B are side views of a stent acting as an external structural feature.

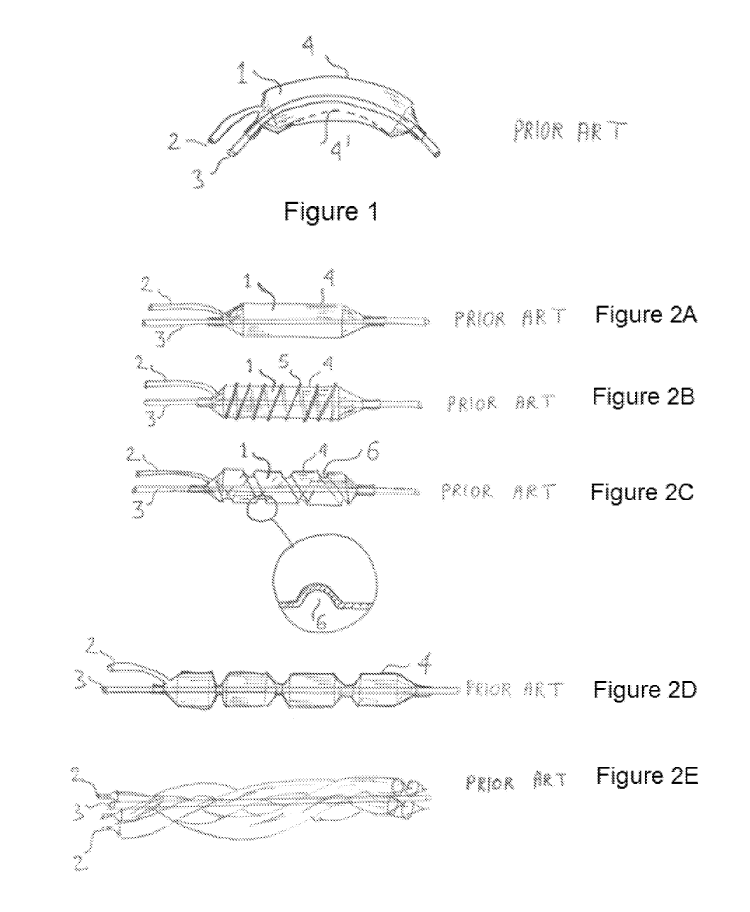

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

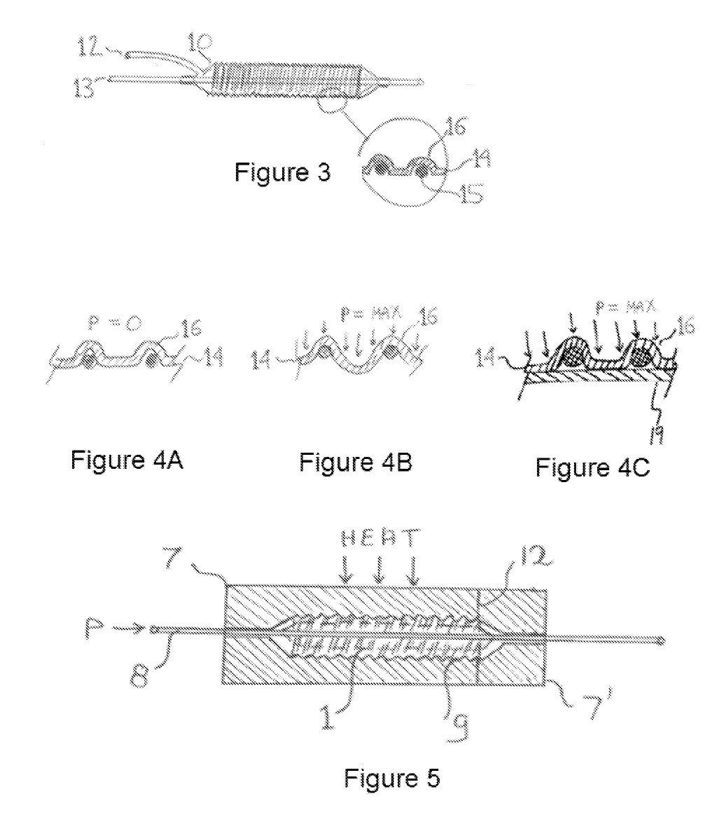

FIG. 3 shows a medical balloon 10 according to an example embodiment. Balloon 10 comprises an inflation tube 12 and optionally incorporates a tube 13 for a guidewire. Balloons according to some embodiments do not incorporate a guidewire tube 13. The wall 14 of balloon 10 is embossed with helical (thread-like) grooves 16 which form a pattern on wall 14. The pitch of grooves 16 may be fixed or may vary along the length of balloon 10. Grooves 16 may comprise a single groove 16 or a plurality of interleaved helical grooves 16 (arranged, for example, like a multi-start thread).

In the illustrated embodiment, grooves 16 cause both the interior surface and the exterior surface of balloon wall 14 to be undulating. In the illustrated embodiment, grooves 16 have depths that are deeper than a thickness of balloon wall 14. The width of the groove is matched to the thickness of the wound filament.

Wall 14 may be made of any suitable material. Example materials for wall 14 include materials such as Nylon12, polyurethane, PET and PEBAX. Wall 14 typically has a thickness in the range of 10 to 30 microns.

A filament 15 made from a high tensile strength material with low compliance is wrapped into groove 16. Filament 15 is affixed within groove 16 in order to prevent separation of filament 15 from wall 14 when balloon 10 is deflated and folded. Filament 15 may, for example, be bonded into groove 16 with a suitable adhesive, embedded in wall 14, or otherwise affixed within groove 16. It is preferable but not mandatory that filament 15 be affixed in groove 16 continuously along its length. In alternative embodiments, filament 15 is affixed in groove 16 at locations that are spaced apart along its length. Filament 15 prevents the pressure inside balloon 10 from flattening groove 16 when balloon 10 is fully inflated.

Filament 15 may comprise a single strand or a plurality of thinner strands. In an example embodiment, filament 15 has a diameter in the range of about 10 microns to about 30 microns. A good choice for the material of filament 15 is Kevlar™. For example, filament 15 may be made from a single strand of Kevlar having a diameter in the above range or a plurality of Kevlar strands each having a smaller diameter.

Other materials may also be used. For example, if a more diametrically compliant balloon is desired a more compliant material can be used for filament 15 such as monofilament nylon or polyester. Filament 15 can be bonded into groove 16, for example using a suitable adhesive. In one embodiment, bonding of filament 15 is performed by diluting a polyurethane adhesive such as TEXIN™ 5265. An example process that may be used for bonding filament 15 into groove 16 and examples of different suitable adhesives and filament materials are given in U.S. Pat. No. 8,430,846 which is hereby incorporated herein by reference for all purposes.

Typical dimensions of groove 16 are depth of 50 μm to 500 μm and a pitch of 50 μm to 2 mm. In general, as the wall thickness of balloon 10 is increased the dimensions (especially depth) of groove 16 should be increased. Typically, the depth and pitch scale with balloon size, where deeper and coarser grooves are used on larger diameter balloons.

A member that connects ends of balloon 10 to one another may be provided inside balloon 10. In the embodiment illustrated in FIG. 3, such a member is provided by guidewire tube 13. The characteristics of the wall of balloon 10 (wall material, wall thickness and dimensions of groove 16) and the connecting member may be adjusted to achieve a balloon having a very small straightening force. If the balloon wall is too flexible, e. g. the pitch of the wound filament is too small, grooves 16 are too deep or the material of wall 14 is too elastic then the balloon can over bend during pressurization because the connecting member (e.g. guidewire tube 13) which does not stretch much, tends to move towards the inner radius of the curve, which is shorter. This allows the outer radius of the balloon to expand and over bend the balloon. The interaction of the connecting member and the too-flexible balloon wall effectively provides a negative straightening force. If a regular balloon has a strong straightening force and a highly embossed balloon with a wound filament has a negative straightening force, it is possible to create a balloon with zero (or very close to zero) straightening force by adjusting the characteristics of the balloon wall. Therefore, the elasticity, embossing pitch and embossing depth can be selected to minimize the straightening force. There is more than one combination that will result in a zero or very small straightening force. There is an advantage in making the guidewire tube somewhat stretchable, as a balloon also stretches a bit under pressure.

In the embodiment of FIG. 3, grooves 16 facilitate allowing the balloon wall on the inner radius of a bend to compress and the balloon wall on the outer radius of the bend to stretch while keeping the balloon cross-section circular. It is best to use a material that has a larger elastic range than standard balloon materials to allow such compression and stretching.

Filament 15 prevents grooves 16 from disappearing even when balloon 10 is fully inflated. Since the pressure within balloon 10 is pushing out the area between the adjacent turns of wound filament 15, increasing the pressure in the balloon tends to increase the depth of grooves 16 rather than flatten them out. This is illustrated in FIGS. 4A and 4B. FIG. 4A shows the wall of an unpressurized balloon and FIG. 4B shows the balloon wall under full pressure. It has been found that in prototypes of such balloons the straightening force can be almost zero at all pressures. Note that a possible embodiment of the disclosed invention could further comprise a sheath or external layer 19 to minimize the undulation of the surface, which can be an undesirable trait in certain circumstances, FIG. 4C.

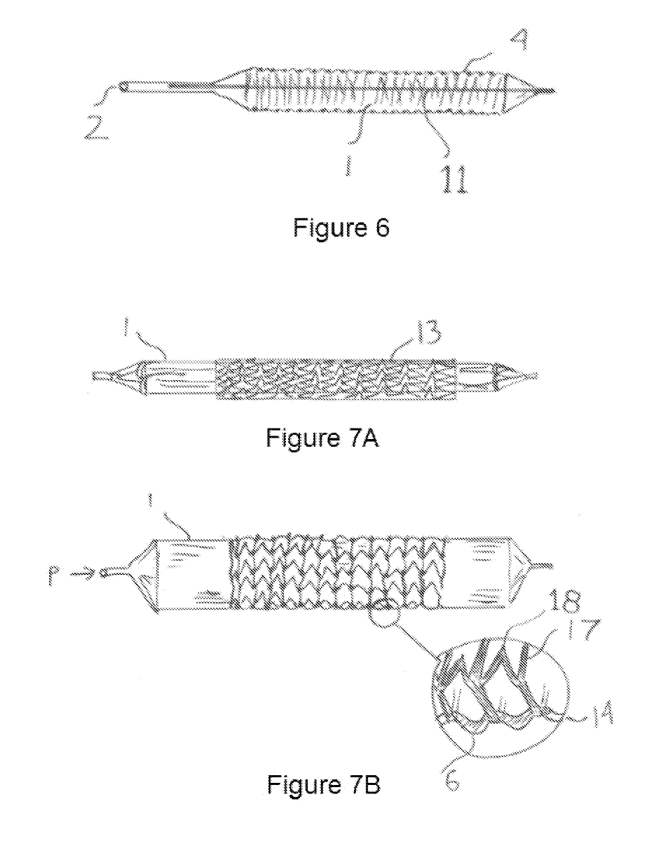

For example, an experimental 6 mm diameter×40 mm long non-compliant balloon made as described herein, with a groove depth and pitch of about 0.4 mm, had over ten times the bending flexibility of a standard balloon (i.e. over 10 times less straightening force) made from the same material and the same thickness. Not only was the straightening force dramatically lower than that of the standard balloon, but the crossing profile was also improved and the burst pressure nearly doubled. Since the wound filament increases the burst strength of the balloon, the balloon wall can be made from a softer and more elastic material than a regular balloon, further decreasing the straightening force. Materials capable of stretching 10% to 50% before breaking are preferred. It was also found that elongation was low, about 10%, and could be eliminated by providing a guidewire tube holding the length nearly constant. For balloons not having a guidewire tube a non-elastic cord can be provided inside the balloon to tie both ends together, as illustrated for example in FIG. 6. Compliance of cord 11 will determine the length compliance of the balloon.

A helical groove 16 may be formed by embossing. Such embossing may be done in conjunction with forming a balloon by methods that are the same as or similar to methods used to manufacture regular balloons. FIG. 5 shows a tube 8 made of a balloon material inserted into a cavity in a mold 7 having a groove pattern 9 machined into its inner wall. Mold 7 is heated, typically to 100-160 degrees C. and tube 8 is expanded into a balloon by pressurizing it. In some embodiments, the balloon is stretched longitudinally at the same time as it is pressurized. After cooling, the balloon can be collapsed by vacuum and removed or the mold can be opened and the balloon removed. Since it is difficult to fabricate the mold from a single piece, a mold split line 12 is provided. For a split line 12 as shown, after cooling, part 7′ is removed and the balloon can be pulled out by using a very slight amount of vacuum or no vacuum at all. If the mold is split longitudinally, the balloon can be removed easily with no need for vacuum but the fit between the mold halves has to be very good in order not to leave a parting line on the finished balloon. Suitable molding machines are commercially available, for example from Interface (www.interfaceusa.com/equipment/balloon-forming-equipment/), and are well known to those of skill in the art.

Using methods of manufacture such as those described above makes it relatively easy to manufacture balloons that have improved properties relative to conventional balloons.

While preferred embodiments use an embossed groove 16 in the form of a single-start thread, mainly for ease of placing filament 15 into groove 16, it should be understood that any form of groove that decreases straightening force is part of the invention. Examples of possible groove patterns include: isolated rings (which may be parallel to one another), multi-start threads, meshes with a steep angle (i.e. from 60° to 90°) relative to the longitudinal axis or any other form.

A different embodiment uses a variable thickness balloon wall to achieve a function like that of a separate filament 15. In such embodiments the balloon is grooved and the balloon wall adjacent to the bottom of each groove is formed to be thicker than parts of the balloon wall between grooves 16. The thickened sections at the bottom of grooves 16 act in the same manner as a filament 15 to prevent grooves 16 from being flattened as the balloon's internal pressure is raised to its maximum working pressure.

Any structural feature preventing the grooved pattern from disappearing when the balloon is pressurized forms part of this invention. For lower pressure balloons it is possible to prevent grooves from disappearing when balloon is pressurized by using a very fine groove pitch and depth. The reason for this is that it takes more force to flatten out a fine pitch grooving pattern than a coarse pitch grooving pattern. For example, the width and depths of grooves may be in the range of 1× to 10× the balloon wall thickness; preferably from 1× to 5× of the wall thickness. For example, where the wall thickness is 30 microns, grooves 16 could have a depth in the range of 30 to 300 microns, preferably 30 to 150 microns.

The following calculation shows how small grooves can be very resistant to being flattened. Assuming the wall thickness is T and the groove radius, measured to the centerline of the thickness is R. Assuming that the groove has a semi-circular cross-sectional shape, the length of the outer layer of the wall from one side of the groove to the other is π(R+0.5T) and the inner wall layer length is π(R−0.5T). After flattening the wall the length of either side is πR, and the stress created in either side of the wall is 0.5πT/πR=T/2R.

This simple calculation shows that as the groove radius R becomes smaller and smaller the stresses and forces needed to create them (which are proportional to the stress in the elastic range), become larger and larger. At some combination of groove radius, wall thickness and pressure, the structure of the grooves themselves without a filament 15 will resist collapse at the working pressure of a balloon. For example, a balloon made of 50 micron Nylon, a groove 100 microns wide and 100 micron deep will hold the pressure of about 5 atm.

Any part of the balloon that is less compliant than the rest of the balloon can be used to resist flattening of grooves at a working pressure of the balloon. For example, a structural feature can be formed in a wall of uniform thickness by increasing the strength of the material in certain areas. It is well known that irradiating many polymers with ionizing radiation causes cross linking and increases the tensile strength. Irradiating a thread-like helix on the balloon will cause the non-irradiated area to bulge out when balloon is pressurized. The irradiation can be done by a UV laser, X-ray machine or gamma rays. The opposite approach can also be used: weakening a helix like (or ring like) pattern on the balloon by laser ablation or mechanical abrasion will cause this are to bulge out when pressurized. A 248 nm excimer laser is an excellent tool for such controlled material removal. An alternate way is heating the balloon in a helical pattern, using a coiled heater, and stretching it, causing the heated areas to become thinner. In such a case the untreated area forms the structural feature.

Some non-limiting enumerated aspects of the invention are as follows:
1. A balloon having a wall with at least one embossed pattern to decrease the straightening force of the balloon. The wall comprises one or more structural features to prevent the embossed patterns from disappearing at any state of inflation within a range of operating pressures for the balloon.
2. A balloon as in aspect 1, wherein the pattern is a helical groove and the structural feature is a wound filament.
3. A balloon as in aspect 1, wherein the structural features are created by strengthening the balloon wall in selected areas.
4. A balloon as in aspect 1, wherein the structural features are created by strengthening the balloon wall in selected areas by irradiation.
5. A balloon as in aspect 1, wherein the structural features are created by strengthening the balloon wall in selected areas by changing the wall thickness.
6. A balloon as in aspect 1, which is manufactured by inflating a heated balloon inside an embossed mold.
7. A balloon as in aspect 1, wherein the lengthening of the balloon is longitudinally restricted.
8. A method for decreasing the straightening force of a balloon, wherein the method comprises: embossing patterns that create grooves in the balloon wall and placing structural features in the grooves.
9. A method as in aspect 8, wherein the grooves are helical and the structural feature is a wound filament.
10. A balloon as in aspect 8, wherein the embossed pattern is helical and the structural features are a wound filament made of Kevlar fiber.
11. A balloon as in aspect 1, wherein the embossed pattern is helical and the structural features are a wound filament bonded to the balloon using an adhesive.
12. A method as in aspect 8, wherein the structural features are created by strengthening the balloon wall in selected areas.
13. A method as in aspect 8, wherein the structural features are created by strengthening the balloon wall in selected areas by irradiation.
14. A method as in aspect 8, wherein the structural features are created by strengthening the balloon wall in selected areas by changing the wall thickness.
15. A method as in aspect 8, of manufacturing the balloon by inflating a heated balloon inside an embossed mold.
16. A method as in aspect 8, wherein the lengthening of the balloon is longitudinally restricted.
17. A balloon having a decreased straightening force when inflated inside at least one external structural feature, wherein the features cause embossed grooves to be formed in the wall of the balloon.
18. A balloon as in aspect 17, wherein external structural feature is a stent.
19. A balloon as in aspect 17, wherein external structural feature is an aortic graft.
20. A balloon having a wall with an embossed pattern to decrease the straightening force, wherein the pattern is capable of withstanding the inflation pressure by having a depth and a pitch of less than 10 times the wall thickness of the balloon.
21. A method of reducing the straightening force of an inflated medical balloon by making the wall of the balloon having a larger elasticity in the longitudinal direction than in the circumferential direction.
22. Apparatus having any new and inventive feature, combination of features, or sub-combination of features as described herein.
23. Methods having any new and inventive steps, acts, combination of steps and/or acts or sub-combination of steps and/or acts as described herein.

### INTERPRETATION OF TERMS

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

“Elastic modulus” is a characteristic of a material and is a ratio of stress to strain in the material. A material having a smaller elastic modulus is easier to stretch than a material having a higher elastic modulus.

“Elastic modulus” is a characteristic of a material and is a ratio of stress to strain in the material. A material having a smaller elastic modulus is easier to stretch than a material having a higher elastic modulus. “Elasticity” is a measure of how easily a material can be elastically deformed. The elasticity of a structure such as a balloon wall in a particular direction (e.g. longitudinal or circumferential) is a measure of how much the structure will expand in the indicated direction when placed under tension in that direction.

“Elastic range” means the range through which a material can be stretched without breaking or undergoing a plastic deformation. Elastic range can be expressed as a percentage. for example, if a 10 cm strip of material can be stretched to 13 cm but any more stretching would result in the material undergoing plastic deformation or breaking then the material can be said to have an elastic range of (13−10)/10×100%=30%.

In this disclosure, “compliance” can be understood to have the same meaning as elasticity. A balloon that is considered “compliant” has a larger elastic range that a “non-compliant” balloon.

“High tensile strength” applied to a material means a material that can withstand high forces without breaking or bursting.

Where a component (e.g. a tube, wall, assembly, mold, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A medical balloon comprising:
a balloon wall of a first material enclosing an interior volume, the balloon wall formed to provide a grooves extending around the balloon wall;
a filament of a second material different from the first material affixed within the groove, the second material having a elastic modulus greater than an elastic modulus of the first material; and
an inflation conduit arranged to provide fluid communication into the interior volume.

2. A medical balloon according to claim 1 wherein the groove extends helically around the balloon.

3. A medical balloon according to claim 2 wherein the balloon wall is formed to provide first and tapered end portions at opposing ends of the balloon and the groove extends in a substantially-continuous helix from the first tapered end portion to the second tapered end portion.

4. A medical balloon according to claim 3 comprising a flexible member extending through the interior volume connecting the first and second end portions.

5. A medical balloon according to claim 2 wherein the groove has a pitch measured longitudinally along an outside of the balloon wall in the range of 50 μm to 2 mm.

6. A medical balloon according to claim 1 comprising first and second end portions at opposing longitudinal ends of the balloon connected by a generally cylindrical portion of the balloon wall where the groove is formed at least in the cylindrical portion of the balloon wall.

7. A medical balloon according to claim 6 comprising a flexible member extending through the interior volume and connecting the first and second end portions to one another.

8. A medical balloon according to claim 7 wherein the flexible member has an axial stiffness greater than that of the balloon wall.

9. A medical balloon according to claim 8 wherein the flexible member comprises a guidewire tube.

10. A medical balloon according to claim 1 wherein the groove has a depth greater than a thickness of the balloon wall.

11. A medical balloon according to claim 1 wherein the groove has a depth in the range of 50 μm to 500 μm.

12. A medical balloon according to claim 1 wherein both inner and outer surfaces of the balloon wall undulate in a plane that cuts across the groove.

13. A medical balloon according to claim 1 wherein the filament comprises a Kevlar fiber.

14. A medical balloon according to claim 1 wherein the filament is adhered in the groove by an adhesive.

15. A medical balloon according to claim 14 wherein the adhesive comprises a polyurethane adhesive.

16. A medical balloon according to claim 1 wherein the first material is selected from the group consisting of Nylon12, PET, polyurethane and PEBAX.

17. A medical balloon according to claim 1 wherein the balloon wall has a thickness in the range of 10 microns to 30 microns.

18. A medical balloon according to claim 1 wherein the groove has a depth less than 10 times a thickness of the material of the wall of the balloon.

19. A medical balloon according to claim 18 wherein the groove is helical and has a width and a width measured longitudinally along an outer surface of the balloon that is less than 10 times the thickness of the material of the wall of the balloon.

20. A medical balloon according to claim 18 wherein the groove remains present under a pressure differential of at least 10 atm between the interior volume and an exterior of the balloon.

21. A medical balloon according to claim 1 wherein the wall of the balloon has a larger elasticity in the longitudinal direction than in the circumferential direction.

22. A method for making a balloon, the method comprising:
forming one or more grooves extending around a generally cylindrical wall portion;
before or after forming the grooves sealing ends of the wall portion to enclose an interior volume of the balloon;
wrapping a filament in the one or more grooves around the wall portion; and
affixing the filament in the one or more grooves.

23. A method according to claim 22 wherein forming the one or more grooves comprises embossing the grooves.

24. A method according to claim 23 wherein embossing the one or more grooves comprises placing the wall portion into a cavity in a mold, the cavity comprising projections corresponding to the one or more grooves and pressurizing an interior of the wall portion.

25. A method according to claim 24 comprising heating the mold.
